(19) United States
(12) Patent Application Publication
(10) Pub. No.: US 2023/0221318 A1
(43) Pub. Date: Jul. 13, 2023

(54) T CELL SPECIFIC BIOMARKERS FOR PREDICTING GRAFT-VS-HOST DISEASE AND HEMATOPOIETIC MALIGNANCY RELAPSE FOLLOWING HEMATOPOIETIC STEM CELL TRANSPLANTATION AND TREATMENT THEREOF

(71) Applicant: Wisconsin Alumni Research Foundation, Madison, WI (US)

(72) Inventors: Nicholas Hess, Cottage Grove, WI (US); Christian Capitini, Madison, WI (US); Kalyan Nadiminti, Madison, WI (US); Peiman Hematti, Madison, WI (US); Jenny Gumperz, Madison, WI (US)

(21) Appl. No.: 18/094,221

(22) Filed: Jan. 6, 2023

Related U.S. Application Data
(60) Provisional application No. 63/297,184, filed on Jan. 6, 2022.

(51) Int. Cl. G01N 33/569

(52) U.S. Cl. CPC G01N 33/56972; G01N 2333/70589; G01N 2800/245

(57) ABSTRACT

The present disclosure generally relates to methods for diagnosing, predicting and treating graft-vs-host disease and/or relapse of a hematologic malignancy following hematopoietic stem cell transplantation based on T-cell specific biomarkers.

This invention was made with government support under CA215461 and TR002375 awarded by the National Institutes of Health. The government has certain rights in the invention.

### FIELD OF THE DISCLOSURE

The present disclosure generally relates to methods for diagnosing, predicting and treating graft-vs-host disease and/or relapse of a hematologic malignancy following hematopoietic stem cell transplantation based on T-cell specific biomarkers.

### BACKGROUND OF THE DISCLOSURE

Despite a growing yet imperfect understanding of the prognostic variables that influence allogeneic hematopoietic stem cell transplantations (allo-HSCT), the field is currently unable to predict an individual patient’s probability of experiencing the two most common negative transplant outcomes, acute graft-vs-host-disease (aGVHD) and hematopoietic malignancy relapse (Adom et al., 2020, Front Immunol 11: 673; Paczesny, 2018, Blood 131: 2193-2204; Chen & Zeiser, 2020, Front Immunol 11: 1854). Allo-HSCT is the standard curative treatment for various hematological malignancies, and while consolidation allo-HSCT has had varying degrees of success (depending on disease type), pre-transplant minimal-residual-disease (MRD) positivity has become an established prognostic marker to identify a patient’s risk of relapsing post-transplant (D′Souza et al., 2020, Biol Blood Marrow Transplant 26: el77-el82; Kanate et al., 2020, Biol Blood Marrow Transplant 26: 1247-1256). However, there are very few tools available currently which can predict disease relapse post-transplant. Donor chimerism measurement is routinely employed as a surrogate to predict relapse and consider pre-emptive treatments, such as reduction of immunosuppression (RI), or pre-emptive donor lymphocyte infusion (DLI), especially for persistently mixed chimerism and/or high-risk malignancies. However, the value and predictability of early chimerism and relapse-risk and survival are controversial and debated (Mountjoy et al., 2020, Leukemia & Lymphoma 62: 252-254). Similarly, presence of full donor chimerism also does not preclude a relapse, especially in the early post-transplant stage. Established prognostic variables for aGVHD risk include HLA (human leukocyte antigen) disparity, conditioning regimen, donor type, and graft source. (Gooptu et al., 2021, Blood 138: 273-282; Anasetti et al., 2012, N. Engl. J. Med. 367: 1487-1496; Gyurkocza & Sandmaier. 2014, Blood 124: 344-353; and Caldemeyer et al., 2017, Biology of Blood and Marrow Transplantation 23: 1989-1997). Although these prognostic variables are well established, it can be difficult to estimate the composite risk at an individual level because a number of these factors can influence the aGVHD outcome.

Both aGVHD and graft-vs-leukemia (GVL) activity, the latter a primary factor in preventing relapse, are mediated by donor T cells that become activated against host and leukemia antigens respectively (Hill et al., 2021, Current Concepts and Advances in Graft-Versus-Host Disease Immunology. Annu Rev Immunol. 39:19-49; Hess et al., 2021, Frontiers in Immunology 12: 3082; Blazar et al., 2020, Nat Rev Clin Oncol. 17: 475-492). Separating these two biological activities has been and remains the most sought-after goal of allo-HSCT research. Novel aGVHD prophylaxis regimens including post-transplant cyclophosphamide have revolutionized the field in terms of aGVHD prevention, especially in mis-matched donor transplantation, but have not reduced the rates of relapse (Martinez-Cibrian et al., 2020, Blood Reviews 100792).

Thus, there remains a need in the art to establish methods for identifying patients that are likely to relapse or develop aGVHD. The disclosure describes methods and systems that address this unmet need.

### SUMMARY OF THE DISCLOSURE

Provided herein are methods of treating a subject at risk of relapse of a hematologic malignancy in a subject in need thereof, comprising selecting the subject at risk of relapse of a hematologic malignancy by determining the number of CD45RO+ T cells in a biosample isolated from the subject; and identifying the subject as at risk of relapse of a hematologic malignancy because the number of CD45RO+ T cells is low in the biosample, wherein this amount is ≤ 200 cells per 0.5 mL of blood; and administering an effective amount of a treatment for the hematologic malignancy to the subject. In certain embodiments, the methods disclosed herein further comprise determining the number of T cells in the biosample isolated from the subject and determining the percentage of Treg cells in the T cells from the sample; and identifying the subject as at risk of relapse of a hematological malignancy when the percentage of Treg cells is reduced to between about 5-14%. In certain embodiments, risk of relapse is assessed by determining that the number of CD45RO+ T cells is low in the biosample, wherein this amount is ≤ 200 cells per 0.5 mL of blood, that the percentage of Treg cells is reduced to between about 5-14%, or that both the number of CD45RO+ T cells is low in the biosample wherein this amount is ≤ 200 cells per 0.5 mL of blood and that the percentage of Treg cells in the biosample is reduced to between about 5-14%.

Also provided herein is a method of predicting relapse of a hematologic malignancy in a subject, the method comprising: determining the number of CD45RO+ T cells in a biosample isolated from the subject, wherein a low number CD45RO+ T cells in the biosample, wherein this amount is ≤ 200 cells per 0.5 mL of blood, predicts relapse of a hematologic malignancy in the subject. In certain embodiments, the methods disclosed herein further comprise determining the percentage of Treg cells in total T cells in the sample, wherein a low percentage of Treg cells predicts relapse of a hematological malignancy in the subject when the percentage of Treg cells is reduced to between about 5-14%. In certain embodiments, relapse of a hematopoietic malignancy is predicted by determining that the number of CD45RO+ T cells is low in the biosample wherein this amount is ≤ 200 cells per 0.5 mL of blood, that the percentage of Treg cells in the sample is reduced to between about 5-14%, or that both the number of CD45RO+ T cells is low in the biosample, wherein this amount is ≤ 200 cells per 0.5 mL of blood, and that the percentage of Treg cells in the biosample is reduced to between about 5-14%.

Also provided herein are methods for treating a subject at risk of acute graft-versus-host disease (aGVHD) in a subject in need thereof, comprising selecting the subject at risk of GVHD by determining the level of CD4+CD8+ double positive T cells (DPT) in a biosample isolated from the subject; determining the level of CD45RO+ T cells in the biosample isolated from the subject; and identifying the subject as at risk of aGVHD because the level of CD4+CD8+ double positive T cells (DPT) is between 4% to 6% of all CD45RO⁺ T cells in the biosample; and administering an effective amount of a treatment for aGVHD to the subject. In certain embodiments these methods further comprise determining in the biosamples the percentage of T cell blasts; the percentage of Treg cells in total T cells; the percentage of CD8+ T cells in total T cells; and the percentage of DPT cells; wherein the subject is treated for aGVHD when the percentage of T cell blasts is about 13-24%, the percentage of Treg cells is about 11-18%, the percentage of CD8+ T cells is about 12-21%, and/or the percentage of DPT cells is about 2.5-4.5%.

Also provided herein are methods for predicting acute graft-versus-host disease (aGVHD) in a subject, the method comprising: determining the level of CD4+CD8+ double positive T cells (DPT) and the level of CD45RO⁺ T cells in a biosample isolated from the subject, wherein the level of CD4+CD8+ double positive T cells (DPT) is between 4% to 6% of all CD45RO⁺ T cells in the biosample predicts acute graft-versus-host disease (aGVHD) in the subject. In certain embodiments these methods further comprise determining in the biosample the percentage of T cell blasts, the percentage of Treg cells in total T cells, the percentage of CD8+ T cells in total T cells, and the percentage of DPT cells, wherein the percentage of T cell blasts is high/elevated, the percentage of Treg cells in total T cells is low/reduced, the percentage of CD8+ T cells in total T cells is high/elevated, and the percentage of DPT cells, wherein aGVHD is precited when the percentage of T cell blasts is about 13-24%, the percentage of Treg cells is about 11-18%, the percentage of CD8+ T cells is about 12-21%, and/or the percentage of DPT cells is about 2.5-4.5%.

Also provided herein are methods for treating a subject at risk of acute graft-versus-host disease (aGVHD) and/or at risk of relapse of a hematologic malignancy in a subject in need thereof, comprising: selecting the subject at risk of GVHD and/or risk of relapse of a hematologic malignancy in a subject by determining the level of CD4+CD8+ double positive T cells (DPT) in a biosample isolated from the subject; determining the level of CD45RO⁺ T cells in the biosample isolated from the subject; identifying the subject as at risk of aGVHD because the level of CD4+CD8+ double positive T cells (DPT) is between 6% to 8% of all CD45RO⁺ T cells in the biosample; and/or identifying the subject as at risk of relapse of a hematologic malignancy because the level of CD4+CD8+ double positive T cells is ≤ 1% of all CD45RO⁺ T cells in the biosample and administering an effective amount of a treatment for aGVHD and/or relapse of a hematologic malignancy to the subject. Certain embodiments of the methods disclosed herein further comprise determining in the biosamples the percentage of T cell blasts; and the percentage of Treg cells in total T cells; wherein the subject is treated for aGVHD when the percentage of T cell blasts is high/elevated, and the percentage of Treg cells is high/elevated. In certain embodiments relapse is treated wherein the number of CD45RO+ T cells is low in the biosample, wherein this amount is≤ 200 cells per 0.5 mL of blood, the percentage of Treg cells is reduced to between about 5-14%, or that both the number of CD45RO+ T cells is low in the biosample, wherein this amount is≤ 200 cells per 0.5 mL of blood, and that the percentage of Treg cells in the biosample is reduced to between about 5-14%.

Also provided herein are methods for predicting acute graft-versus-host disease (aGVHD) and/or relapse of a hematologic malignancy in a subject, the method comprising; determining the level of CD4+CD8+ double positive T cells (DPT) and the level of CD45RO⁺ T cells in a biosample isolated from the subject, wherein the level of CD4+CD8+ double positive T cells (DPT) is between 6% to 8% of all CD45RO⁺ T cells in the biosample predicts acute graft-versus-host disease (aGVHD) in the subject and wherein the level of CD4+CD8+ double positive T cells is ≤ 1% of all CD45RO⁺ T cells in the biosample predicts relapse of a hematologic malignancy in the subject. Certain embodiments of the methods disclosed herein further comprise determining in the biosample the percentage of T cell blasts and the percentage of Treg cells in total T cells, wherein when the percentage of T cell blasts is high/elevated and the percentage of Treg cells in total T cells is low/reduced aGVHD and/or relapse of a hematological malignancy is predicted in the subject. In certain embodiments, acute graft-versus-host disease (aGVHD) and/or relapse of a hematologic malignancy is predicted wherein the number of CD45RO+ T cells is low in the biosample, wherein this amount is ≤ 200 cells per 0.5 mL of blood, that the percentage of Treg cells is reduced to between about 5-14%, or that both the number of CD45RO+ T cells is low in the biosample, wherein this amount is ≤ 200 cells per 0.5 mL of blood, and that the percentage of Treg cells in the biosample is reduced to between about 5-14%.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIGS. 1A-1B show the number of samples collected during the study disclosed in Example 1. FIG. 1A shows that collection intervals were divided into 4-day increments between day 0 and 29 and six-day increments between day 30 and 99. Dashed line represents the average number of samples collection in each interval from 35 patients. FIG. 1B highlights the number of samples collected for each patient with the average number of 12 samples/patient denoted by the dashed line.

FIG. 2 shows the flow cytometric gating strategy. Sequential gating strategy was used on all patient samples across all the collection intervals.

FIGS. 3A-3F show the analysis of effector and memory (CD45RO+) T cell population dynamics. FIG. 3A shows the quantified number of CD3⁺/CD45RO⁺ effector and memory T cell populations in 0.5 mL of blood for each allo-HSCT outcome group from day 0 to day 100. FIG. 3B shows the data points from day 5 to day 22 post-transplant (up to four collection intervals or data points per subject) graphed in their respective outcome group. Geometric mean and 95% confidence intervals are shown above each column with the significance value from a non-parametric t-test indicated above each respective pairing. FIG. 3C shows receiver-operator-characteristic (ROC) analysis for each comparison showing significance in FIG. 3B. The area under the curve (AUC) and p-value are shown. FIG. 3D through FIG. 3F show the analysis from FIGS. 3A-3C repeated using data points relative to the timeframe when each subject received their highest GVHD score (data points from day 27, 33, 40, 47, 54, 61, 68, 75 and 83 were used for the relapse and No GVHD groups). Only data points from -2, -1 and 0 weeks from highest GVHD score were used for the analysis in E-F. NS = not significant.

FIG. 4A through FIG. 4F show the analysis of double positive (CD4⁺/CD8β⁺) T cell population dynamics. FIG. 4A shows the quantified number of CD3⁺/CD45RO⁺/CD4^{+/}CD8β⁺ double positive T cell population in 0.5 mL of blood for each allo-HSCT outcome group from day 0 to day 100. FIG. 4A shows the data points from day 5 to day 22 post-transplant (up to four collection intervals or data points per subject) graphed in their respective outcome group. Mean and 95% confidence intervals are shown above each column with the significance value from a parametric t-test indicated above each respective pairing. FIG. 4C shows the receiver-operator-characteristic (ROC) analysis performed for each comparison showing significance in (FIG. 4B). The area under the curve (AUC) and p-value are shown. FIG. 4E and FIG. 4F show the analysis from FIG. 4A through FIG. 4C repeated using data points relative to the timeframe when each subject received their highest GVHD score (data points from day 27, 33, 40, 47, 54, 61, 68, 75 and 83 were used for the relapse and No GVHD groups). Only data points from -2, -1 and 0 weeks from highest GVHD score were used for the analysis in E-F. NS = not significant.

FIG. 5 shows the analysis of pro-inflammatory co-stimulatory proteins in effector and memory (CD3⁺/CD45RO⁺) T cell populations. The co-stimulatory protein target is listed above each respective graph. Bar are separated by T cell population and by collection interval. Graphs are ordered (left to right and top to bottom) based on the expression density of each protein.

FIG. 6 shows the analysis of anti-inflammatory inhibitory proteins in effector and memory (CD3⁺/CD45RO⁺) T cell populations. The inhibitory protein target is listed above each respective graph. Bar are separated by T cell population and by collection interval. Graphs are ordered (left to right and top to bottom) based on the expression density of each protein.

FIG. 7 illustrates the cytometric analyses set forth herein that predict relapse and acute graft versus host disease (aGVHD) in hematopoietic stem cell bone marrow transplant patients over a time course of 97 days post-transplant. The top row of the figure illustrates repeated days of blood collection and analysis. The center row provides illustrations of results of flow cytometry gated using antibodies for five T cell markers (from left to right): CD45RA- vs. CD45RO+ cells; T cell blasts gated for CD3+/CD45RO+ cells; Treg cells gated for CD4+ and CD25+; CD8+ cells gated for CD4+ and CD8β; and double positive T cells gated for CD4+ and CD8+ markers. The bottom row provides a color-coding scheme for individuals with relapse of hematological malignancy relapse (purple); no relapse (yellow); G0 levels of aGVHD (blue) vs. G1 levels of aGVHD (orange); and G1 levels of aGVHD (green) vs. G2 levels of GVHD (red).

FIG. 8 illustrated flow cytometry patterns of T cells gated using antibodies for five T cell markers illustrated in FIG. 7, bar graphs illustrating data from 417 hematopoietic stem cell bone marrow transplant patients as discussed in the Examples herein, showing the data points from day 7 to day 17 post-transplant (up to three collection intervals or data points per subject) graphed in their respective outcome group, wherein geometric mean and 95% confidence intervals are shown above each column with the significance value from a non-parametric t-test indicated above each respective pairing; and ROC analysis is shown for each comparison displaying significance, wherein area under the curve (AUC) and p-values are shown. The bar graphs are arranged in the same order as the representative individuals in FIG. 7, from left to right purple (P), yellow (Y), blue (B), orange (O), green (G), and red (R).

FIG. 9 shows multivariate analysis for combinations of flow cytometric analyses informative for hematological malignancy relapse or aGVHD for patient blood samples taken between 7-17 days post-transplant (top row) and patient blood samples taken between 22-84 days post-transplant, showing that the number of CD45RO+ T cells and % of Treg cells are informative for hematological malignancy relapse 7-17 post-transplant and cells characterized by these markers plus percentage of DPT cells are informative 22-84 days post-transplant (PT, lefthand graphs). GVHD is predicted by cytometric analyses gated for % of T cell blasts (CD3+), % of Treg cells (CD4T), % of CD8+ cells, and % of DPT cells (7-17 days PT), whereas GVHD is predicted by % of CD8+ cells and % of DPT cells 22-84 days PT (middle graphs). GVHD having severity ≥ G2 is predicted by % of Treg cells and % of DPT cells 7-17 day PT and by % CD8+ cells and % DPT cells 22-84 days PT (right-hand graphs).

FIG. 10 shows results of a predictive algorithm, that integrates cytometric data with the five cytological markers to a single output, illustrated for two patients (036 and 037) anonymously coded to protect their identities. These results, showing likelihood of relapse, nominal or GVHD are shown in the bottom row of the figure with the frequency information for each outcome set forth in the graphs above.

### DETAILED DESCRIPTION OF THE DISCLOSURE

The present disclosure generally relates to methods for diagnosing, predicting and treating graft-vs-host disease and/or relapse of a hematologic malignancy following hematopoietic stem cell transplantation based on T-cell specific biomarkers.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In particular embodiments disclosed herein is a method of treating a subject at risk of relapse of a hematologic malignancy in a subject in need thereof, the method comprising: selecting the subject at risk of relapse of a hematologic malignancy by determining the number of CD45RO+ T cells in a biosample isolated from the subject; and identifying the subject as at risk of relapse of a hematologic malignancy because the number of CD45RO+ T cells is low in the biosample; and administering an effective amount of a treatment for a hematologic malignancy to the subject.

In certain embodiments, the methods disclosed herein further comprise determining the number of T cells in the biosample isolated from the subject and determining the percentage of Treg cells in the T cells from the sample; and identifying the subject as at risk of relapse of a hematological malignancy when the percentage of Treg cells is low/reduced. As set forth herein, the percentage of Treg cells is low or reduced when present in a biosamples at about 11-18% of T cells in the sample.

In some embodiments disclosed herein is a method of treating a subject at risk of acute graft-versus-host disease (aGVHD) in a subject in need thereof, comprising: selecting the subject at risk of aGVHD by determining the level of cluster of differentiation 4 (CD4)+cluster of differentiation 8 (CD8)+ double positive T cells (DPT) in a biosample isolated from the subject; determining the level of CD45RO⁺ T cells in a biosample isolated from the subject; and identifying the subject as at risk of aGVHD because the level of CD4+CD8+ double positive T cells (DPT) is between 4% to 6% of all CD45RO⁺ T cells in the biosample; and administering an effective amount of a treatment for aGVHD to the subject.

In certain embodiments, the methods disclosed herein further comprise determining the percentage of Treg cells in total T cells in the sample, wherein a low percentage of Treg cells predicts relapse of a hematological malignancy in the subject. As set forth herein, the percentage of Treg cells is low or reduced when present in a biosamples at about 11-18% of T cells in the sample.

In particular embodiments disclosed herein is a method of treating a subject at risk of aGVHD and/or at risk of relapse of a hematologic malignancy in a subject in need thereof, comprising: selecting the subject at risk of aGVHD and/or risk of relapse of a hematologic malignancy in a subject by determining the level of CD4+CD8+ double positive T cells (DPT) in a biosample isolated from the subject; determining the level of CD45RO+ T cells in a biosample isolated from the subject; identifying the subject as at risk of aGVHD because the level of CD4+CD8+ DPT is between 6% to 8% of all CD45RO⁺ T cells in the biosample; and/or identifying the subject as at risk of relapse of a hematologic malignancy because the level of CD4+CD8+ DPT cells is ≤ 1% of all CD45RO⁺ T cells in the biosample and administering an effective amount of a treatment for aGVHD and/or relapse of a hematologic malignancy to the subject.

In certain embodiments these methods further comprise determining in the biosamples the percentage of T cell blasts; the percentage of Treg cells in total T cells; the percentage of CD8+ T cells in total T cells; and the percentage of DPT cells; wherein the subject is treated for aGVHD when the percentage of T cell blasts is high/elevated, the percentage of Treg cells is high/elevated, the percentage of CD8+ T cells is high/elevated, and/or the percentage of DPT cells is high/elevated.

In these embodiments, the percentage of T cell blasts is “high” or “elevated” when present in a biosample at about 13-24% of T cells in the sample. As set forth herein when the percentage of Treg cells is low or reduced when present in a biosamples at about 11-18% of T cells in the sample. As set forth herein when the percentage of CD8+ T cells is “high” or “elevated” when present in a biosample at about 12-21% of T cells in the sample. As set forth herein the percentage of DPT cells in the sample is “low” or “reduced” when present in a biosample at about 2.5-4.5% of T cells in the sample.

In some embodiments disclosed herein is a method of predicting relapse of a hematologic malignancy in a subject, the method comprising; determining the number of CD45RO+ T cells in a biosample isolated from the subject, wherein a low number CD45RO+ T cells in the biosample predicts relapse of a hematologic malignancy in the subject.

In certain embodiments these methods further comprise determining in the biosample the percentage of T cell blasts, the percentage of Treg cells in total T cells, the percentage of CD8+ T cells in total T cells, and the percentage of DPT cells, wherein the percentage of T cell blasts is high/elevated, the percentage of Treg cells in total T cells is low/reduced, the percentage of CD8+ T cells in total T cells is high/elevated, and the percentage of DPT cells is high/elevated.

In these embodiments, the percentage of T cell blasts is “high” or “elevated” when present in a biosample at about 13-24% of T cells in the sample. As set forth herein when the percentage of Treg cells is low or reduced when present in a biosamples at about 11-18% of T cells in the sample. As set forth herein when the percentage of CD8+ T cells is “high” or “elevated” when present in a biosample at about 12-21% of T cells in the sample. As set forth herein the percentage of DPT cells in the sample is “low” or “reduced” when present in a biosample at about 2.5-4.5% of T cells in the sample.

In some embodiments disclosed herein is a method of predicting aGVHD in a subject, the method comprising; determining the level of CD4+CD8+ double positive T cells (DPT) and the level of CD45RO⁺ T cells in a biosample isolated from the subject, wherein the level of CD4+CD8+ DPT is between 4% to 6% of all CD45RO⁺ T cells in the biosample predicts aGVHD in the subject.

Certain embodiments of the methods disclosed herein further comprise determining in the biosamples the percentage of T cell blasts; and the percentage of Treg cells in total T cells; wherein the subject is treated for aGVHD when the percentage of T cell blasts is high/elevated, and the percentage of Treg cells is high/elevated. In other embodiments risk for more serious aGVHD is identified in a subject by determining the percentage of DPT cells and the percentage of Treg cells, wherein the subject is at risk for aGVHD when the percentage of Treg cells is high/elevated, and the percentage of DPT cells is high/elevated.

Also provided herein are methods for predicting relapse of a hematologic malignancy and/or onset of aGVHD. In particular embodiments disclosed herein is a method of predicting aGVHD and/or relapse of a hematologic malignancy in a subject, the method comprising; determining the level of CD4+CD8+ double positive T cells (DPT) and the level of CD45RO⁺ T cells in a biosample isolated from the subject, wherein the level of CD4+CD8+ DPT is between 6% to 8% of all CD45RO⁺ T cells in the biosample predicts aGVHD in the subject and wherein the level of CD4+CD8+ DPT cells is ≤ 1% of all CD45RO⁺ T cells in the biosample predicts relapse of a hematologic malignancy in the subject.

Certain embodiments of the methods disclosed herein further comprise determining in the biosample the percentage of T cell blasts and the percentage of Treg cells, wherein when the percentage of T cell blasts is high/elevated and the percentage of Treg cells in total T cells is low/reduced, aGVHD and/or relapse of a hematological malignancy is predicted in the subject.

Graft-vs-host disease (GVHD) can be classified as acute or chronic GVHD. Acute GVHD is characterized by selective damage to organs and tissues including, but not limited to, the liver, skin (rash), mucosa, and gastrointestinal (GI) tract. Acute GVHD is staged as follows: overall grade (skin-liver-gut) with each organ staged individually from a low of 1 to a high of 4. Grade I(A) GVHD is characterized as mild disease, grade II(B) GVHD as moderate, grade III(C) as severe, and grade IV(D) life-threatening. Chronic GVHD also attacks the above organs, but over its long-term course is also known to cause damage to the lungs, connective tissue, eyes and exocrine glands. In particular embodiments, the methods disclosed herein can be used to diagnose, predict or treat acute GVHD. In particular embodiments, the methods disclosed herein can be used to diagnose, predict or treat chronic GVHD.

In particular embodiments, the subject has undergone a hematopoietic stem cell transplantation. A hematopoietic stem cell transplantation refers to procedures that restore stem cells that were destroyed.

As used herein biosample refers to a sample obtained from the subject. In particular embodiments, the biosample is tissue, whole blood or plasma.

The biosamples used in the methods disclosed herein can be collected at various times following transplantation. In some embodiments, the biosample is collected from the subject at about day 0 to about day 100 after transplant. In some embodiments, the biosample of the disclosure is collected from the subject at about day 5 to about day 22 after transplant (peri-transplant period). In some embodiments, the biosample is collected from the subject at about day 25 to about day 60 after transplant. In some embodiments, the biosample is collected from the subject at least 22 days after transplant.

The expression of CD45RA is generally associated with naive T cells (CD45RA⁺). T cells, which express CD45RO antigen (CD45RO⁺), are called either effector cells or memory cells and proliferate in response to an antigen. Naive T cells lose the CD45RA antigen after activation and begin to express CD45RO. In particular embodiments, the levels or numbers of CD45RO⁺ T cells are characterized as low when they are at or below 50 cells per 0.5 mL of blood during the first 100 days after transplant. In particular embodiments, the levels or numbers of CD45RO⁺ T cells are characterized as low when they are at or below 100 cells per 0.5 mL of blood during the first 100 days after transplant. In particular embodiments, the levels or numbers of CD45RO⁺ T cells are characterized as low when they are at or below 200 cells per 0.5 mL of blood during the first 100 days after transplant. In particular embodiments, the levels or numbers of CD45RO⁺ T cells are characterized as low when they are at or below 500 cells per 0.5 mL of blood during the first 100 days after transplant. In particular embodiments, the methods involve determining the level or number of CD45RO⁺ in a subject and then comparing the level or number to a reference level or range. Typically, the reference level is representative of the number or value of CD45RO⁺ in a persons or tissues that have not relapsed following a hematopoietic stem cell transplantation and whose clinical prognosis data are available.

In particular embodiments, a subject is identified as at risk of aGVHD when the level of CD4+CD8+ double positive T cells (DPT) is between 4% to 6% of all CD45RO⁺ T cells in the biosample. In particular embodiments, a subject is identified as at risk of aGVHD when the level of CD4+CD8+ DPT is between 6% to 8% of all CD45RO⁺ T cells in the biosample. In particular embodiments, a subject is identified as at risk of aGVHD when the level of CD4+CD8+ DPT is 4%, 5%, 6%, 7%, 8%, 9%, 10%, 11%, 12%, 13%, 14%, 15%, 16%, 17%, 18%, 19% or 20 % of all CD45RO⁺ T cells in the biosample.

In certain further embodiments, a subject is identified as being at risk of aGVHD when the percentage of T cell blasts is high/elevated, the percentage of Treg cells is low/reduced, the percentage of CD8β⁺ T cells is high/elevated, and the percentage of DPT cells is high/elevated.

In particular embodiments, a subject is identified as at risk of a hematologic malignancy when the level of CD4+CD8+ double positive T cells (DPT) is less than 1% of all CD45RO⁺ T cells in the biosample. In particular embodiments, a subject is identified as at risk of a hematologic malignancy when the level of CD4+CD8+ DPT is 0.1%, 0.2%, 0.3%, 0.4%, 0.5%, 0.6%, 0.7%, 0.8%, 0.9%, or 1% of all CD45RO⁺ T cells in the biosample.

In certain further embodiments, a subject is identified as at risk of a hematologic malignancy when the number of T cells in a sample and the percentage of Treg cells are elevated.

In particular embodiments, determining the number of CD45RO+ T cells comprises flow cytometry. In particular embodiments, determining the level of CD4+CD8+ double positive T cell the level of CD4+CD8+ DPT comprises flow cytometry. In particular embodiments the number of T cell blasts is determined by detected CD3+ T cells by flow cytometry. In particular embodiments the percentage of Treg cells is determined by detected CD4+ T cells by flow cytometry.

The term “subject” is intended to include human and non-human animals, particularly mammals.

In some embodiments, the methods disclosed herein relate to treating a subject for a relapse of a hematologic malignancy. In some embodiments, the methods disclosed herein relate to treating a subject for graft-vs-host disease (GVHD) following a hematopoietic stem cell transplantation. In some embodiments, the subject received post-transplant GVHD prophylactic treatment such as cyclophosphamide (PTCy), Cyclosporine, Tacrolimus, Sirolimus, Methotrexate (MTX), Corticosteroids and/or Mycophenolate Mofetil (MMF).

The terms “treatment” or “treat” as used herein refer to both therapeutic treatment and prophylactic or preventative measures. Those in need of treatment include subjects having a hematologic malignancy and/or GVHD as well as those prone to having a hematologic malignancy and/or GVHD. In some embodiments, the hematological malignancy is a cancer of the blood and blood-forming organs (bone marrow and lymphoid tissues) such as leukemia, lymphoma and plasma cell dyscrasia. In some embodiments, the hematological malignancy is Angioimmunoblastic T-cell lymphoma, Acute Myeloid Leukemia, Philadelphia chromosome (Ph)-negative B cell acute lymphoblastic leukemia (ALL), T-cell ALL, Myelodysplastic syndromes, Hodgkin lymphoma, chronic myeloid leukemia, Early T-cell Precursor (ETP) ALL, Philadelphia Chromosome positive B cell ALL, Blastic plasmacytoid dendritic cell neoplasm , Juvenille myelomonocytic leukemia, or Diffuse large B-cell lymphoma. In some embodiments, the methods disclosed herein can be used to treat relapse of a hematologic malignancy and/or GVHD.

In particular embodiments, a subject identified as at risk for relapse is treated by donor lymphocyte infusion. In particular embodiments, a subject identified as at risk for relapse is treated by stopping the use of all aGVHD prophylaxis drugs.

In particular embodiments, a subject identified as at risk or having GVHD is treated with a standard prophylaxis drug such as cyclosporine, tacrolimus, MMF, MTX, and/or Sirolimus. In particular embodiments, a subject identified as at risk or having GVHD can be treated with a standard aGVHD treatment such as corticosteroid like methylprednisolone. In particular embodiments, a subject identified as at risk or having steroid-refractory aGVHD is treated with a combination of methylprednisolone and ruxolitinib.

The terms “administration” or “administering” as used herein refer to providing, contacting, and/or delivering a compound or compounds by any appropriate route to achieve the desired effect. Administration can include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, or using implants.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of a compound or composition capable of inducing a desired therapeutic effect.

The terms “pharmaceutically acceptable carrier” or “physiologically acceptable carrier” as used herein refer to one or more formulation materials suitable for accomplishing or enhancing the delivery of a compound or composition capable of inducing a desired therapeutic effect.

When used for in vivo administration, the formulations of the disclosure should be sterile. The formulations of the disclosure can be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

The formulations can be presented in unit dosage form and can be prepared by any method known in the art of pharmacy. Actual dosage levels of the active ingredients in the formulation of the present disclosure can be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose can also depend on the route of administration. For example, subcutaneous administration can require a higher dosage than intravenous administration.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only, and should not be construed as limiting the scope of the invention in any way.

### Example 1: Analysis of T Cell Metrics in Allo-HSCT Patients

### Methods

### Subjects

All patients receiving an allogeneic hematopoietic stem cell transplantation (allo-HSCT) at the University of Wisconsin-Madison were prospectively enrolled between October 2020 and August 2021. A total of 35 patients were enrolled on this study. Patient characteristics are outlined in Table 1. Eligible patients had to receive post-transplant cyclophosphamide for GVHD prophylaxis at day +3/+4. Conditioning regimen, HLA matching status, graft source and patient disease type were all non-exclusion criteria for this study.

TABLE 1

Subject Transplantation Variables

| Conditioning Regimen | Donor Matching | Graft Source |
|---|---|---|
| MAC = 21/34 (62%) | 8/8 MRD = 4/34 (12%) | PBSC = 33/34 (97%) |
| RIC = 2/34 (6%) | 8/8 MUD = 18/34 (53%) | BM = 1/34 (3%) |
| NMA = 11/34 (32%) | 4/8 HP = 9/34 (26%) | |
| | ⅞ MMUD = 2/34 (9%) | |

### Sample Collection

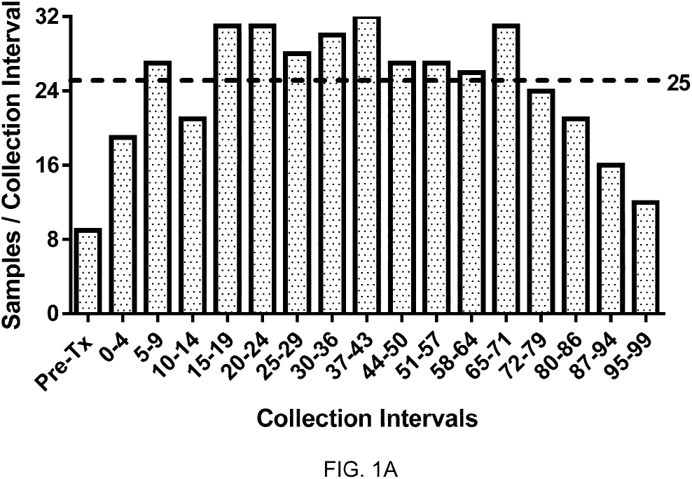

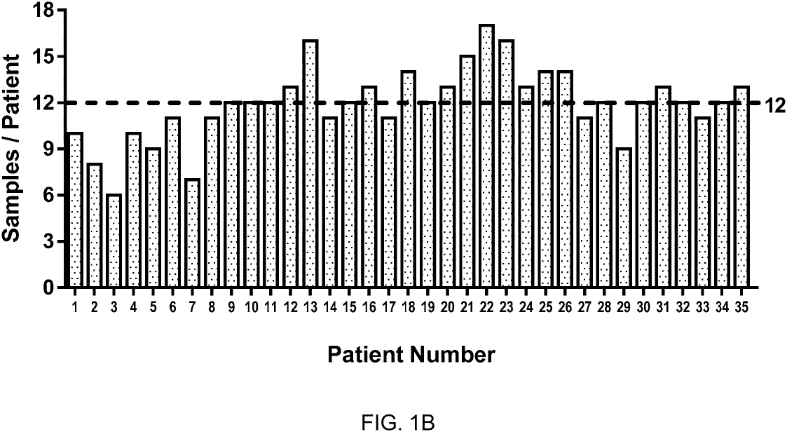

All allo-HSCT patients received daily blood draws while they were in-patient and weekly blood draws when they become out-patient. Excess unused blood from study patients were set aside after initial use and stored at 4° C. for 3-10 days before processing. The goal of the study was to process a single patient’s blood sample in every four days interval starting at day 0 (e.g. between days 0-4, 5-9 and 10-14 days post-transplant) for the first 29 days and then in every six-day interval starting at day 30 and ending at day 99. This resulted in 16 possible collection intervals. Collection results for each patient are highlighted in FIG. 1. On average, blood samples from 12 of the 16 collection intervals were collected.

### Sample Processing

A total of 1.5 mL of blood was aliquoted from each patient collection tube, centrifuged at 600xg for 4 min at 4° C. and the resulting plasma collected and stored at -150° C. for future analysis. The cell pellet was resuspended in 1.5 mL of 1X RBC lysis buffer (BioLegend), vortexed and rested for 2 min prior to the addition of 1 mL of PBS and centrifugation as before. Supernatant was disposed of and a second round of RBC lysis was performed. Afterwards, the cell pellet was re-suspended in 1.5 mL of flow buffer (IX PBS + 2% FBS) and 500 mL aliquoted into three flow cytometry tubes for staining.

### Flow Cytometry Staining/Panels

As stated above, after processing each patient sample was divided into three tubes, each stained with a different panel of flow cytometric antibodies. Each panel of antibodies were added together to create a master mix prior to staining. Approximately 13.5 µL of each master mix was added to each sample for staining (~1.5µL per antibody). All flow cytometric antibodies were from BioLegend unless stated otherwise.

Panel 1 was used for all 35 patients and consisted of antibodies for: CD45RA, CD4, CD69, panγδ (Miltenyi), CD45RO, CD8β (Ebioscience), CD25, panHLA, CD3. Panel 2a was used for patients 1-14 and consisted of antibodies for: ICOS, CD4, OX40, PD-1, CD45RO, CD8β (Ebioscience), 4-1BB, PanHLA and CD3. Panel 2b was used for patients 15-24 and consisted of antibodies for: FASL, CD4, CD27, NKG2D, CD45RO, CD8β (Ebioscience), CD28, PanHLA and CD3. Panel 2c was used for patients 25-35 and consisted of antibodies: LAG3, CD4, CD44, TIGIT, CD45RO, CD8β (Ebioscience), CTLA4, PanHLA and CD3. Panel 3 was used for all 35 patients and consisted of antibodies for: isotype, CD4, isotype, isotype, CD45RO, CD8β (Ebioscience), isotype, PanHLA and CD3. The isotype control was matched to the corresponding expression marker from panel 2a, 2b or 2c whenever possible. For all panels, the fluorophore order was as follows: PB/BV421, BV510, FITC/AF488, PE, PerCPCy5.5, PECy7, APC/AF647, AF700 and APC/Fire.

### Analysis

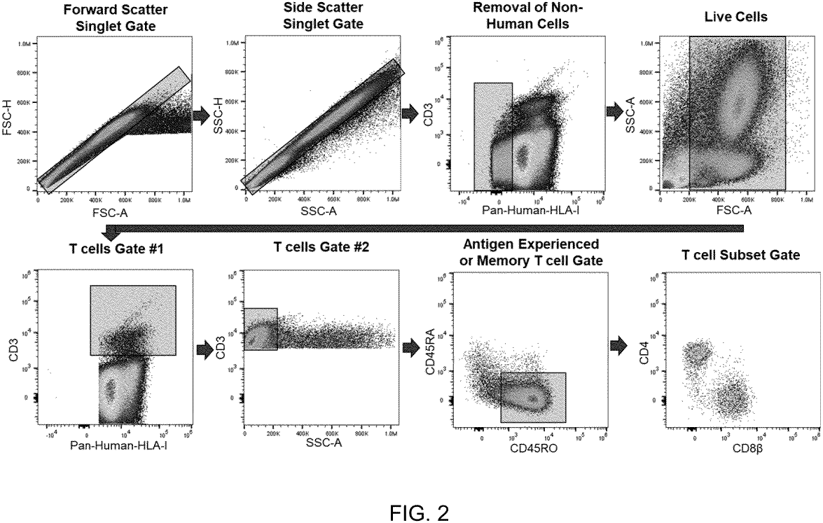

All flow samples were analyzed on FlowJo v10.8.1 software. Samples were gated using the strategy outlined in FIG. 2. Data from FlowJo was then transferred to GraphPad Prism 6 (or newer) for analysis. Statistical tests used in the analysis included parametric and non-parametric t-tests for the analysis of linear (i.e. percentages) and logarithmic (i.e. cell quantification) analysis. Receiver-operator-characteristic (ROC) analysis was also performed to determine efficacy as a predictive biomarker.

With a minimum follow-up of 120 days, patients were then divided into five respective categories based on their allo-HSCT outcome. The categories are relapse, healthy, grade 1 aGVHD, grade 2 aGVHD and grade 3-4 aGVHD. Patient assignment into each respective aGVHD category was based on their highest aGVHD grade achieved between day 0 and 100. The development of acute or chronic GVHD (cGVHD) after day 100 was not factored into this analysis. Relapse after day 100 was included into this analysis. One patient was excluded from the analysis due to the development of grade 2 aGVHD at day 76 and relapse of disease at day 167. One patient developed severe cGVHD symptoms at day 77 and was included in the grade 2 aGVHD category. A breakdown of category is included in Table 2.

TABLE 2

Patient Outcomes After Allo-HSCT w/PTCy

| Relapse | No Relapse or aGVHD | Grade 1 aGVHD | Grade 2 aGVHD | Grade 3-4 aGVHD |
|---|---|---|---|---|
| 5/34 (15%) | 10/34 (29%) | 6/34 (18%) | 9/34 (26%) | 4/34 (12%) |

### Results

### Patient Cohort and Sample Collection

A total of 35 patients were prospectively enrolled onto the study with the majority receiving myeloablative conditioning (62%) followed by non-myeloablative (32%) and a minority receiving reduced-intensity conditioning (6%). The most common donor:recipient matching criteria was 8/8 matched unrelated donors (53%) followed by haploidentical donors (26%), 8/8 matched related donors (12%) and ⅞ mismatched unrelated donors (9%). All but one of the patients received G-CSF mobilized peripheral blood as their hematopoietic stem cell source (Table 1).

A total of 417 samples were collected for this study with an average of 12 samples per patient and 25 samples per collection interval (FIG. 1). With a minimum of 120 days of follow-up, the rates of relapse and severe aGVHD (grade 3-4) were all within the normal range. The most common outcome for patients was a relapse-free/aGVHD-free outcome (29%) followed by grade 2 aGVHD (26%), grade 1 aGVHD (18%), relapse (12%) and severe grade 3-4 aGVHD (12%) (Table 2). One of the 35 patients was excluded from the analysis due to the development of grade II aGVHD at day 78 and relapse at day 168. A second patient with T cell acute lymphoblastic leukemia (T-ALL) who relapsed at day 60 had all their data points beyond day 44 censored due to the possibility of conflicting data from the T-ALL.

### Gating Strategy

A highly conservative gating strategy was performed to avoid unnecessary and confounding outliers that could skew the analysis. First, single cells were collected through a forward (FSC-H vs FSC-A) and side (SCC-H vs SSC-A) scatter singlet gate (FIG. 2A). Second, to confirm live human cells were analyzed, non-pan-HLA-I expressing cells were excluded and low FSC-A/high SSC-A “dying” cells were removed (FIG. 2B). Next, T cells were gated through two sequential gates. The first was a CD3 vs pan-HLA-I gate followed by a CD3 vs SSC-A gate to clearly separate them from other lymphocytes (non-CD3 expressers) and granulocytes which have high SSC-A (FIG. 2C). The T cells were then gated on CD45RO⁺ cells followed by CD4⁺, DPT⁺, CD8⁺ and DN populations. T_{reg} populations were defined as CD4⁺/CD25⁺. “Blasting” is a term to define proliferating cells characterized by a high FSC-A. In this analysis, blasting was determined by any T cell having higher than a 500k reading by FSC-A (dependent on flow cytometer FSC voltage).

### Analysis of Effector and Memory T Cell Populations

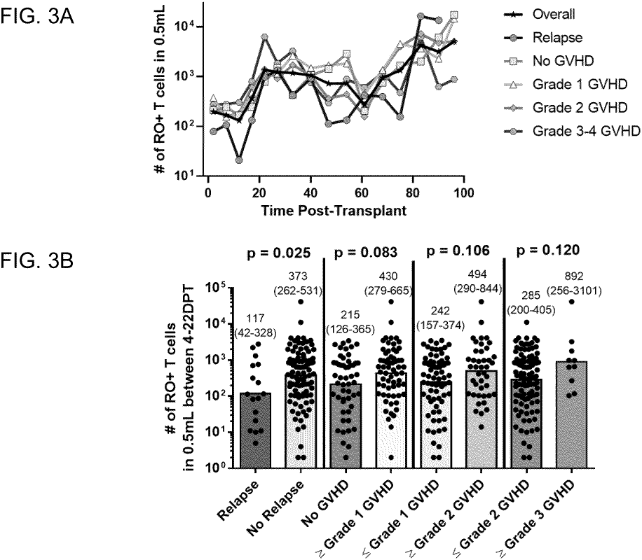

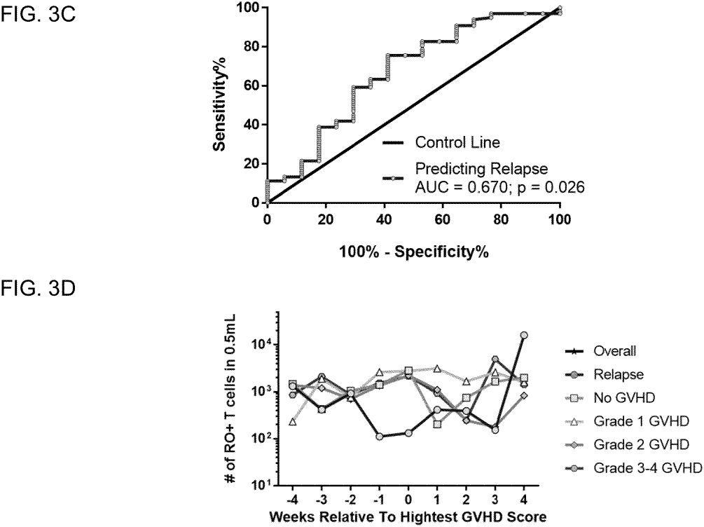

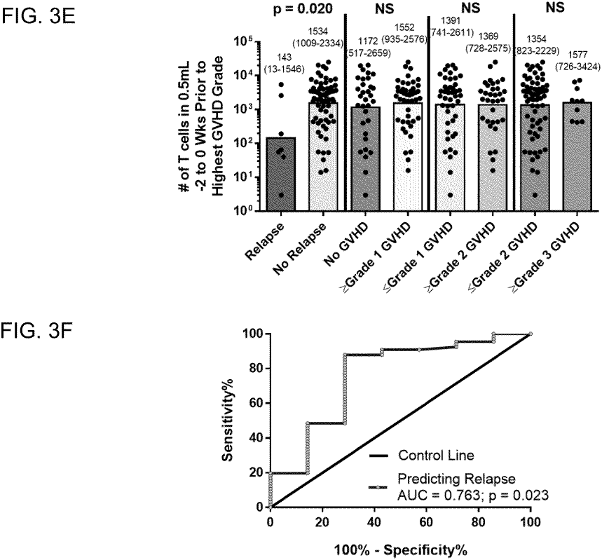

Investigation into quantification of CD45RO⁺ T cells after allo-HSCT with post-transplant cyclophosphamide revealed that a substantial number of T cells were present in blood immediately following transplantation. This population of T cells remained stable for the first 20 days, after which there was an approximately 10-fold expansion of the number of T cells in the blood. This expansion, which occurred between days 15-24 and peaked at ~1E3 T cells/0.5 mL of blood was followed by a slight contraction phase that lasted until day 60 and reduced the number of CD45RO⁺ T cells to ~250 T cells/0.5 mL of blood. After this contraction phase, another expansion phase occurred that facilitated a steady expansion of CD45RO⁺ T cells, ending at ~4E3 at day 100 (FIG. 3A).

In the early peri-transplant period, described as between day 5-22 post-transplant, there was a general trend toward increased overall CD45RO⁺ T cell numbers in the groups containing aGVHD patients (FIG. 3B). This trend was also present when the aGVHD patients were stratified by their highest aGVHD score (FIG. 3B). There were a statistically significant lower number of CD45RO⁺ T cells in the patients that went on to relapse compared to the remaining cohort of patients (p=0.025). This extended into an ROC analysis suggesting that lower CD45RO⁺ T cell numbers in the peri-transplant period was predictive of patient relapse with an AUC of 0.670 and a p-value of 0.026 (FIG. 2C).

A second analysis taking into account the collection intervals prior to a patient’s highest aGVHD score revealed few differences in the number of CD45RO⁺ T cells in the blood of patients except for relapse patients (FIG. 3D). While this analysis was hindered by the lack of data points in the relapse outcome condition, it nonetheless highlighted that the contraction phase (day 25-60) in patients who would eventually relapse was more severe than in the rest of the cohort (FIG. 3A). This equated to having a significantly reduced CD45RO⁺ T cell population compared to patients that didn’t relapse between day 40 and 54 and that these lower T cell levels was a predictive biomarker of later relapse in these patients (FIGS. 3E-F).

### Analysis of the DPT Population

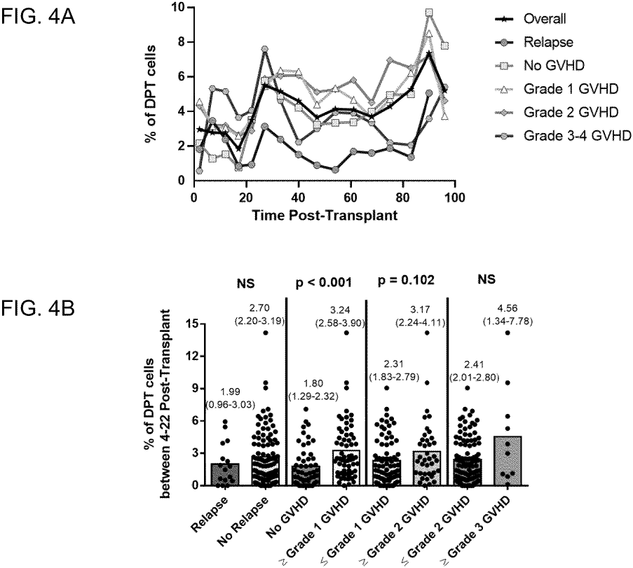

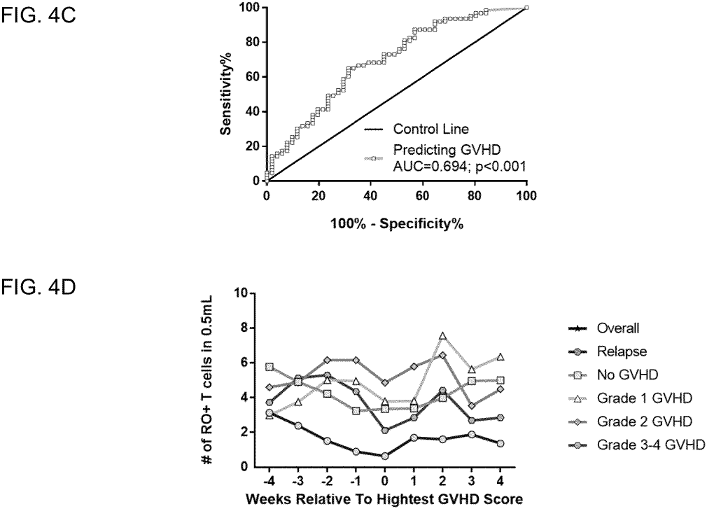

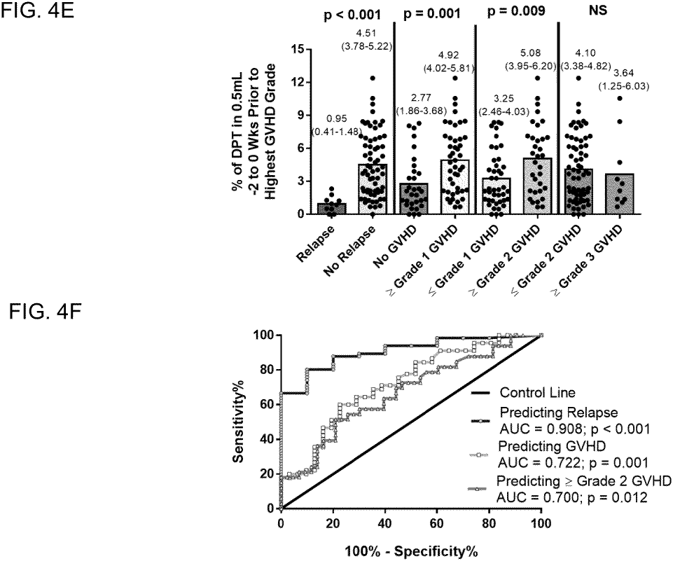

The differentiation of DPT in the patient cohort during the peri-transplant period was highly predictive of developing ≥ grade 1 aGVHD (FIG. 4A through FIG. 4C). In general, the percentage of DPT was stable in patients between day 2 and 17 at ~3% of all CD45RO⁺ T cells (FIG. 4A). This was followed by an expansion of the DPT population around day 22 to between 4-6%, which remained relatively stable during the remaining 78 days (FIG. 4A). During this peri-transplant period, there was no difference in the percentage of DPT between relapse and non-relapsing patients or patients with various grades of aGVHD. The percentage of DPTs was highly predictive of patients developing any grade of aGVHD with an ROC analysis yielding an AUC of 0.694 and a p-value of less than 0.001 (FIG. 4B and FIG. 4C).

Surprisingly, the continued tracking of the DPT frequency after day 22 revealed that DPT were a highly significant predictive biomarker of both relapse and developing ≥ grade 2 aGVHD (FIGS. 4D-F). In general, most aGVHD patients experienced an increase in the percentage of DPT in the blood in the two collection periods prior to being diagnosed with their highest grade of aGVHD (FIG. 4D). Additionally, relapse patients had a sustained lower level of DPT in their blood (~1%) compared to patients remaining cancer-free (4.5%) (FIG. 4E). Thus, analyzing the frequency of DPTs in the blood after the peri-transplant period resulted in a highly significant predictive biomarker of both relapse and the development of ≥ grade 2 aGVHD (FIG. 4F).

### T Cell Populations Expression Profiles

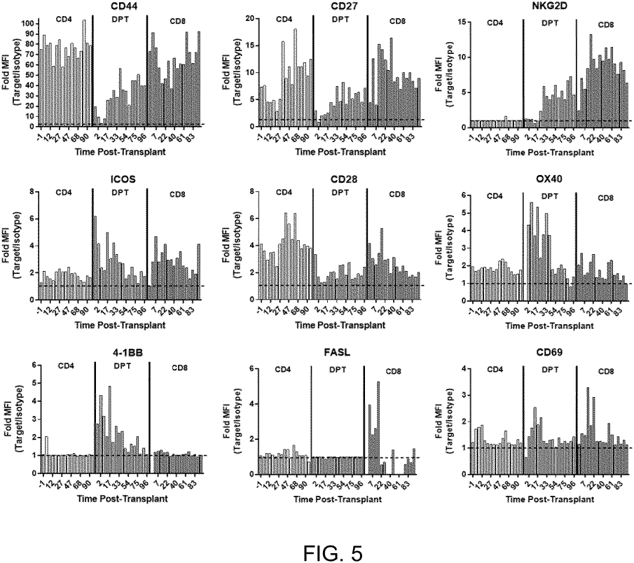

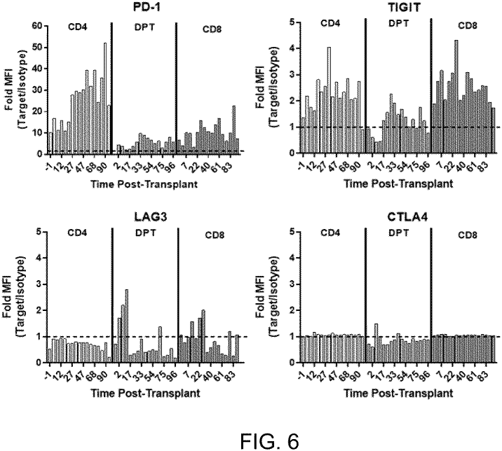

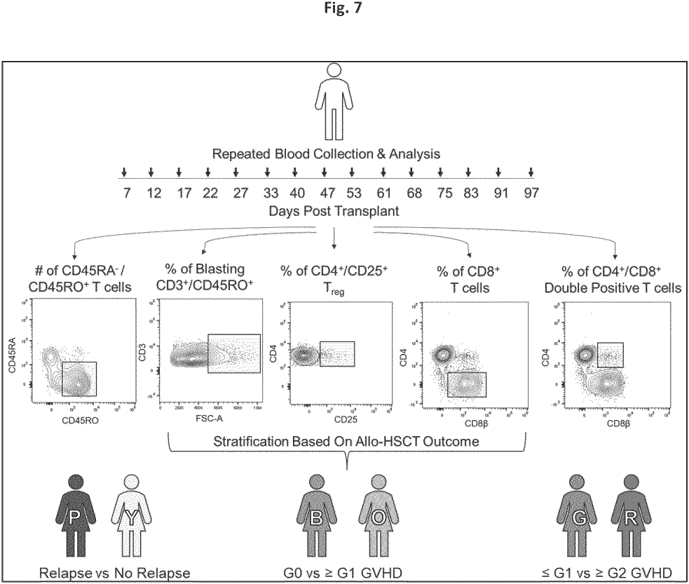

Analysis of nine different co-stimulatory proteins and four inhibitory proteins on the surface of CD4, DPT and CD8 T cells revealed strikingly different expression patterns between these T cell lineages and over the course of the first 100 days post-transplant (FIGS. 5-6). The CD4 lineage were the highest expressors of both CD44, CD27, CD28 and PD-1. In general, the expression of CD27, CD28 and PD-1 also increased over time while CD44 expression remained constant (FIG. 5 and FIG. 6). In comparison, the CD8 lineage were the highest expressors of NKG2D, FASL and TIGIT. The expression of FASL was overall low and only appeared early after transplant. Meanwhile NKG2D expression increased until ∼day 60 after which it began to decrease. The expression of TIGIT was the only other inhibitory protein expressed on these three T cell populations of which it was fairly constant across all three populations (FIG. 5 and FIG. 6).

The DPT expression profile was strikingly different then its CD4 and CD8 counterparts. It had an overall lower expression of CD44, CD27, CD28, PD-1 and TIGIT. Furthermore, it was the only T cell population to express OX40 and 4-1BB at appreciable levels. The DPT population was also marked by intermediate levels of NKG2D and the equivalent levels of ICOS as the CD8 population (FIG. 5 and FIG. 6). No T cell population consistently expressed CD69, LAG 3 or CTLA4 (FIG. 5 and FIG. 6).

Cytometric analyses were further performed to predict relapse and acute graft versus host disease (aGVHD) in hematopoietic stem cell bone marrow transplant patients over a time course of 97 days post-transplant. These experiments are illustrated in FIG. 7, wherein the top row of the figure illustrates repeated days of blood collection and analysis. Flow cytometry was performed as set forth in FIG. 7 using antibodies for five T cell markers as described above and shown (from left to right): CD45RA- vs. CD45RO+ cells; T cell blasts gated for CD3+/CD45RO+ cells; Treg cells gated for CD4+ and CD25+; CD8+ cells gated for CD8β+; and double positive T cells gated for CD4+ and CD8β+ markers.

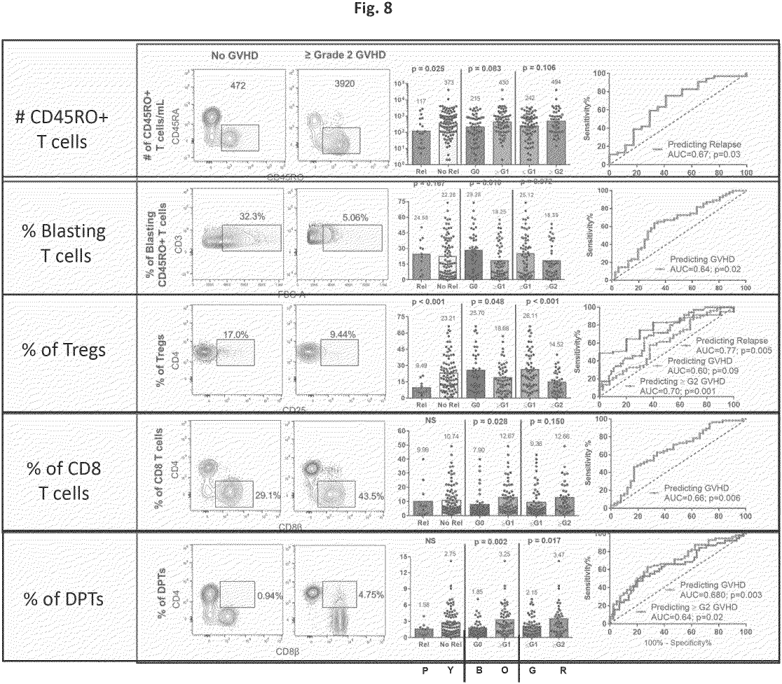

The results of flow cytometry experiments are exemplified in FIG. 8 and the data produced thereby rendered in bar graphs and multivariate analysis curves, with the statistical significance of the results. As is seen in the Figure, the number of CD45RO+ cells is greater in subjects not showing relapse of hematological malignancy and somewhat elevated in patients with aGVHD (although with lower statistical significance). The multivariate analysis shown in the curve over a time course of 0 to 100 days post-transplant (PT). The percent of T cell blasts is also greater in patients not showing relapse over the time course, but are reduced in patient biosamples undergoing aGVHD, with greater suppression associated with greater severity (G2 vs. G1. The percent of Treg cells is greater in patients not showing relapse of hematopoietic malignancy with high statistical significance (p<0.001) and these cell numbers are lower in patients with aGVHD, again with greater reductions being associated with greater disease severity (and with statistical significance of p<0.001). The percent CD8β+ cells has no correlation with relapse of hematopoietic malignancy but greater percentages of these cells are associated (albeit weakly) with aGVHD. Finally, the percentage of double positive T cells (CD4+/CD8+) has no significant correlation with relapse of hematopoietic malignancy but shows statistical significance (p<0.002) with aGVHD.

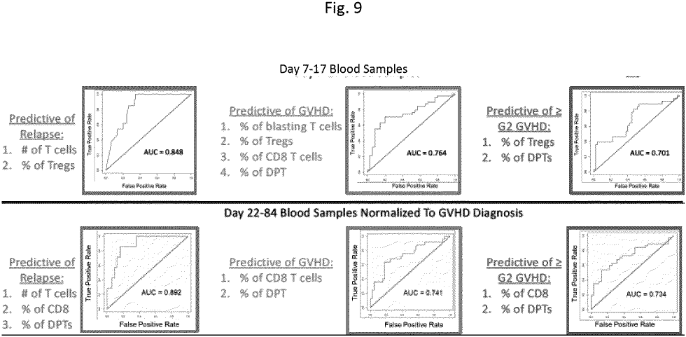

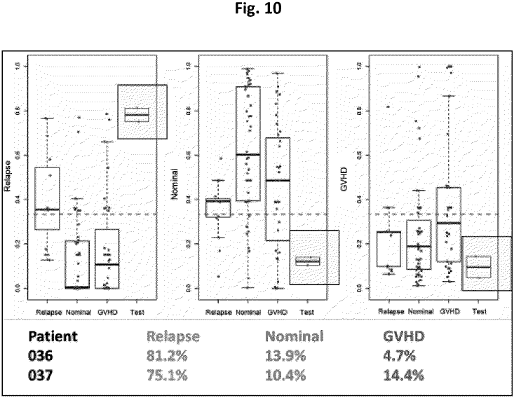

The results of multivariate analysis and the predictions that can be made thereby are shown in FIG. 9 for combinations of flow cytometric analyses informative for hematological malignancy relapse or aGVHD for patient blood samples taken between 7-17 days post-transplant (top row) and patient blood samples taken between 22-84 days post-transplant (bottom row). These results show that the number of T cells and % of Treg cells are informative for hematological malignancy relapse 7-17 post-transplant and cells characterized by these markers plus percentage of DPT cells are informative 22-84 days post-transplant (PT, left-hand graphs). aGVHD is predicted by cytometric analyses gated for % of T cell blasts (CD3+), % of Treg cells (CD4+), % of CD8β+ cells, and % of DPT cells (7-17 days PT), whereas GVHD is predicted by % of CD8+ cells and % of DPT cells 22-84 days PT (middle graphs). GVHD having severity ≥ G2 is predicted by % of Treg cells and % of DPT cells 7-17 days PT and by % CD8β+ cells and % DPT cells 22-84 days PT (right-hand graphs).

These analyses can be adapted to predict hematological malignancy relapse or aGVHD in individual patients, as shown in FIG. 10. Using a predictive algorithm, that integrates cytometric data with the five cytological markers to a single output, the Fig. shows the likelihood of relapse, nominal or GVHD for two patients (036 and 037) anonymously. These results are shown in the bottom row of the figure with the frequency information for each outcome set forth in the graphs above.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present invention.

What is claimed is:

1. A method of treating a subject at risk of relapse of a hematologic malignancy in a subject in need thereof, the method comprising:
(a) selecting the subject at risk of relapse of a hematologic malignancy by
(i) determining the number of CD45RO+ T cells in a biosample isolated from the subject; and
(ii) identifying the subject as at risk of relapse of a hematologic malignancy because the number of CD45RO+ T cells is low in the biosample; and
(b) administering an effective amount of a treatment for the hematologic malignancy to the subject.

2. The method of claim 1, wherein the number of CD45RO+ T cells in the biosample are determined using flow cytometry.

3. The method of claim 1, wherein the subject has undergone hematopoietic stem cell transplantation.

4. The method of claim 3, wherein the biosample is obtained from day 5 to day 22 after the hematopoietic stem cell transplantation.

5. The method of claim 1, wherein the number of CD45RO+ T cells is low when the number of CD45RO+ T cells is ≤ 200 cells per 0.5 mL of blood.

6. The method of claim 3, wherein the biosample is obtained from day 25 to day 60 after the hematopoietic stem cell transplantation.

7. The method of claim 6, wherein the number of CD45RO+ T cells is low when the number of CD45RO+ T cells is ≤ 200 cells per 0.5 mL of blood.

8. The method of claim 1, wherein the biosample is tissue, whole blood or plasma.

9. The method of claim 1, wherein the treatment comprises a donor lymphocyte infusion.

10. A method of predicting relapse of a hematologic malignancy in a subject, the method comprising: determining the number of CD45RO+ T cells in a biosample isolated from the subject, wherein a low number CD45RO+ T cells in the biosample predicts relapse of a hematologic malignancy in the subject.

11. The method of claim 10, wherein the number of CD45RO+ T cells is low when the number of CD45RO+ T cells is ≤ 200 cells per 0.5 mL of blood.

12. A method of treating a subject at risk of acute graft-versus-host disease (aGVHD) in a subject in need thereof, comprising:
(a) selecting the subject at risk of aGVHD by
(i) determining the level of CD4+CD8+ double positive T cells (DPT) in a biosample isolated from the subject;
(ii) determining the level of CD45RO+ T cells in the biosample isolated from the subject; and
(iii) identifying the subject as at risk of aGVHD when the level of CD4+CD8+ double positive T cells (DPT) is between 4% to 6% of all CD45RO⁺ T cells in the biosample; and
(b) administering an effective amount of a treatment for aGVHD to the subject.

13. The method of claim 12, wherein the level of CD4+CD8+ double positive T cell in the biosample is determined using flow cytometry.

14. The method of claim 12, wherein the subject has undergone hematopoietic stem cell transplantation.

15. The method of claim 14, wherein the biosample is obtained from day 5 to day 22 after hematopoietic stem cell transplantation.

16. The method of claim 12, wherein the biosample is tissue, whole blood or plasma.

17. The method of claim 12, wherein the treatment comprises a prophylaxis drug and/or methylprednisolone or ruxolitinib.

18. A method of predicting acute graft-versus-host disease (aGVHD) in a subject, the method comprising: determining the level of CD4+CD8+ double positive T cells (DPT) and the level of CD45RO⁺ T cells in a biosample isolated from the subject, wherein the level of CD4+CD8+ DPT is between 4% to 6% of all CD45RO⁺ T cells in the biosample predicts aGVHD in the subject.

9. A method of treating a subject at risk of acute graft-versus-host disease (aGVHD) and/or at risk of relapse of a hematologic malignancy in a subject in need thereof, comprising:
(a) selecting the subject at risk of aGVHD and/or risk of relapse of a hematologic malignancy in a subject by
(i) determining the level of CD4+CD8+ double positive T cells (DPT) in a biosample isolated from the subject;
(ii) determining the level of CD45RO+ T cells in the biosample isolated from the subject;
(iii) identifying the subject as at risk of aGVHD because the level of CD4+CD8+ DPT is between 6% to 8% of all CD45RO⁺ T cells in the biosample; and/or identifying the subject as at risk of relapse of a hematologic malignancy because the level of CD4+CD8+ DPT cells is ≤ 1% of all CD45RO⁺ T cells in the biosample and
(b) administering an effective amount of a treatment for aGVHD and/or relapse of a hematologic malignancy to the subject.

20. The method of claim 19, wherein the level of CD4+CD8+ double positive T cells (DPT) in the biosample is determined using flow cytometry.

21. The method of claim 19, wherein the subject has undergone hematopoietic stem cell transplantation.

22. The method of claim 21, wherein the biosample is obtained from at least 22 days after hematopoietic stem cell transplantation.

23. The method of claim 19, wherein the biosample is tissue, whole blood or plasma.

24. The method of claim 19, wherein the treatment comprises a prophylaxis drug, and/or methylprednisolone and/or ruxolitinib for aGVHD and/or donor lymphocyte infusion for the treatment of relapse.

25. A method of predicting acute graft-versus-host disease (aGVHD) and/or relapse of a hematologic malignancy in a subject, the method comprising; determining the level of CD4+CD8+ double positive T cells (DPT) and the level of CD45RO⁺ T cells in a biosample isolated from the subject, wherein the level of CD4+CD8+ DPT is between 6% to 8% of all CD45RO⁺ T cells in the biosample predicts aGVHD in the subject and wherein the level of CD4+CD8+ DPT is ≤ 1% of all CD45RO⁺ T cells in the biosample predicts relapse of a hematologic malignancy in the subject.

26. The method of claim 1 further comprising
(c) determining the number of T cells in the biosample isolated from the subject and determining the percentage of Treg cells in the T cells from the sample; and
(d) identifying the subject as at risk of relapse of a hematological malignancy when the percentage of Treg cells is low/reduced.

27. The method of claim 11 further comprising determining the percentage of Treg cells in total T cells in the sample, wherein a low percentage of Treg cells predicts relapse of a hematological malignancy in the subject.

28. The method of claim 27, wherein the percentage of Treg cells is about 11-18%.

29. The method of claim 12, further comprising
(c) determining in the biosamples:
(i) the percentage of T cell blasts;
(ii) the percentage of Treg cells in total T cells;
(iii) the percentage of CD8β+ T cells in total T cells; and
(iv) the percentage of DPT cells;
wherein the subject is treated for aGVHD when the percentage of T cell blasts is high/elevated, the percentage of Treg cells is high/elevated, the percentage of CD8β+ T cells is high/elevated, and/or the percentage of DPT cells is high/elevated.

30. The method of claim 29 further comprising determining in the biosample the percentage of T cell blasts, the percentage of Treg cells in total T cells, the percentage of CD8β+ T cells in total T cells, and the percentage of DPT cells, wherein the percentage of T cell blasts is high/elevated, the percentage of Treg cells in total T cells is low/reduced, the percentage of CD8β+ T cells in total T cells is high/elevated, and the percentage of DPT cells is high/elevated.

31. The method of claim 30, wherein the subject is treated for acute graft-versus-host disease (aGVHD) when the percentage of T cell blasts is about 13-24%, the percentage of Treg cells is about 11-18%, the percentage of GD8β+ T cells is about 12-21%, and/or the percentage of DPT cells is about 2.5-4.5%.

32. The method of claim 19, further comprising
(c) determining in the biosamples:
(i) the percentage of T cell blasts; and
(ii) the percentage of Treg cells in total T cells;
wherein the subject is treated for aGVHD when the percentage of T cell blasts is high/elevated, and the percentage of Treg cells is high/elevated.

33. The method of claim 32 further comprising determining in the biosample the percentage of T cell blasts and the percentage of Treg cells in total T cells, wherein when the percentage of T cell blasts is high/elevated and the percentage of Treg cells in total T cells is low/reduced aGVHD and/or relapse of a hematological malignancy is predicted in the subject.

34. The method of claim 33, wherein the subject at risk of acute graft-versus-host disease (aGVHD) and/or at risk of relapse of a hematologic malignancy is treated when the percentage of T cell blasts is about 13-24%, and the percentage of Treg cells is about 11-18%.
